(19) United States
(12) Patent Application Publication
(10) Pub. No.: US 2018/0220976 A1
(43) Pub. Date: Aug. 9, 2018

(54) MEDICAL PROCEDURE GUIDANCE SYSTEM AND METHOD OF OPERATION

(71) Applicant: Whale Imaging, Inc., Waltham, MA (US)

(72) Inventors: Xingbai He, Belmont, MA (US); Changguo Ji, Lexington, MA (US); Xun Zhu, Waltham, MA (US)

(21) Appl. No.: 15/426,791

(22) Filed: Feb. 7, 2017

(51) Int. Cl. A61B 6/08; A61B 6/00; G01T 1/24; A61B 34/20

(52) U.S. Cl. CPC A61B 6/08; A61B 6/487; A61B 6/488; A61B 6/5205; A61B 2034/2065; A61B 6/4225; G01T 1/247; A61B 34/20; A61B 2034/2068; A61B 6/461

(57) ABSTRACT

A system and method are provided for performing fluoroscopic procedures with assistance of a laser created guide line to reduce a reliance on radiation creating imaging devices during the procedure. The system and method reduce an amount of radiation exposure to patients and care providers during procedures that require assisted imaging. Specifically, an automated laser guidance system and method of use is provided to reduce fluoroscopic radiation, reduce operation time, and increase operative accuracy.

### FIELD OF THE INVENTION

The present invention relates to a method and system for providing guidance for medical procedures. In particular, the present invention relates to method and system for performing fluoroscopic surgical procedures with assistance of a guiding laser beam mapped from a plan line overlaid onto a fluoroscopic preview image and onto a surface of a subject patient to improve procedure efficiencies and accuracies for orthopedic surgeons, as well as reduce radiation exposure of the subject patient.

### BACKGROUND

Generally, fluoroscopy is considered indispensable during contemporary orthopedic surgery. However, there is increasing concern regarding occupational safety in the operating room (OR). Orthopedic surgeons are increasingly using X-ray based fluoroscopic techniques in the operation theatre or in the fluoroscopy room. Procedures such as kyphoplasty, vertebroplasty, deformity correction, pelvic fixation, intramedullary inter-locking nails and computerized tomography (CT) guided biopsies require radiation exposure. Vertebroplasty and kyphoplasty are similar medical spinal procedures in which bone cement is injected through a small hole in the skin percutaneously into a fractured vertebra with the goal of relieving back pain caused by vertebral compression fractures. The general philosophy followed by most healthcare facilities is to minimize radiation dosages such that that all radiation exposures must be justified and that the doses must be kept “as low as reasonably achievable” (ALARA). The overall use of radiation in procedures performed by orthopedic surgeons (e.g., fluoroscopy) is not as much as that used by interventional cardiologists. However, there is increasing concern regarding occupational safety in the operating room (OR). In particular, during a course of a career, an orthopedic surgeon and their OR staff could be exposed to potentially dangerous cumulative levels of radiation. This long term exposure can cause substantial cytogenetic and chromosomal damage, potentially increasing cancer risk. Even relatively small doses of radiation should be considered dangerous over the long-term. Therefore, it is accepted within the industry that annual exposure should be kept to an absolute minimum. Additionally, protective measures, including observance of safe working distance from the radiation source and the routine use of protective garments, have been established.

Another common technique used in orthopedic surgeries is the utilization of Kirschner Wires (K-wires) during a medical procedure. In particular, K-wires are one of the mostly widely used tools in fixing bone fractures in orthopedic surgery. K-wire is also often used during the pre-operative and intro-operative planning, such as aligning the nail direction of the surgery through the single or multiple views of X-ray projective imaging. In a conventional procedure, the surgeon inserts a dedicated K-wire guide wire to confirm position under image intensification multiple times before drilling and tapping. This procedure not only adds to expensive operative time, but also inevitably introduces additional dose to both surgeons and patients.

### SUMMARY

There is a need for improvements for guiding medical procedures, including fluoroscopic-guided procedures, in particular, to reduce an amount of radiation exposure to patients and care providers during procedures that require assisted imaging and guidance. The present invention is directed toward further solutions to address this need, in addition to having other desirable characteristics. Specifically, an automated laser guidance system is provided to reduce overall fluoroscopic radiation, reduce operation time, and increase operative accuracy. In particular, the present invention provides a system and method for a laser guidance system to be used in connection with intra-operative fluoroscopic imaging during procedures that rely on the assistance of imaging devices.

### BRIEF DESCRIPTION OF THE FIGURES

These and other characteristics of the present invention will be more fully understood by reference to the following detailed description in conjunction with the attached drawings, in which:

FIG. 1A is a diagrammatic illustration of a prior art system;

FIG. 1B is an exemplary depiction of an x-ray fluoroscopy system for use in accordance with the present invention;

FIGS. 2A and 2B are exemplary depictions of a laser guidance system with rotary laser-diodes and their planetary locus around fluoroscopic imager, in accordance with the present invention;

FIGS. 3A and 3B are exemplary depictions of a laser guidance system with laser-diodes and their line locus in two directions on fluoroscopic imager, in accordance with the present invention;

FIGS. 4A and 4B are exemplary depictions of one or more surgical guide lines planned on fluoroscopic preview image, in accordance with the present invention;

FIGS. 5A and 5B are exemplary depictions of one or more laser diodes positioned and aligned according to guide lines planned on fluoroscopic preview image, in accordance with the present invention;

FIG. 6 is an illustrative flowchart depicting operation of a laser guidance system, in accordance with aspects of the invention; and

FIG. 7 is a diagrammatic illustration of a high level architecture for implementing processes in accordance with aspects of the invention.

### DETAILED DESCRIPTION

An illustrative embodiment of the present invention relates to an imaging system and method of operation for use in conjunction with a fluoroscopic imaging procedure implemented as guidance during a medical procedure. In particular, the present invention relates to an automated planetary laser guidance system with one or more rotary laser-diodes for use in conjunction with a fluoroscopic imager. The laser guidance system provides one or more guiding laser beams directed onto a subject to provide guidance during an operation. The guiding laser beams are automatically positioned and aligned after a user (e.g., a surgeon) makes plan lines through fluoroscopic preview images. As a result, the present invention helps surgeons to complete efficient and accurate pre-operative and intra-operative planning and with less exposure to fluoroscopic radiation during the procedure. Use of the present invention reduces an amount of exposure experienced by the patient and the operating staff because the surgeon can rely on the guiding laser beams for guidance rather than having to utilize a radiation causing imager throughout a duration of a procedure.

Specifically, prior to starting the procedure, surgeons utilize imaging to create one or more plan lines to be directed by the guide laser system onto a subject as guiding laser beams. The surgeon creates the plan lines on a fluoroscopic preview image provided on a software interface. The plan lines created by the surgeon are recorded by the laser guidance system to be used for positioning of the guiding laser beams directed onto the subject during the procedure. The directed guiding laser beams are created by one of a plurality of laser diodes. The laser guidance system positions and rotates the laser diodes to provide the guiding laser beams directed onto the subject body during the intra-operative surgery.

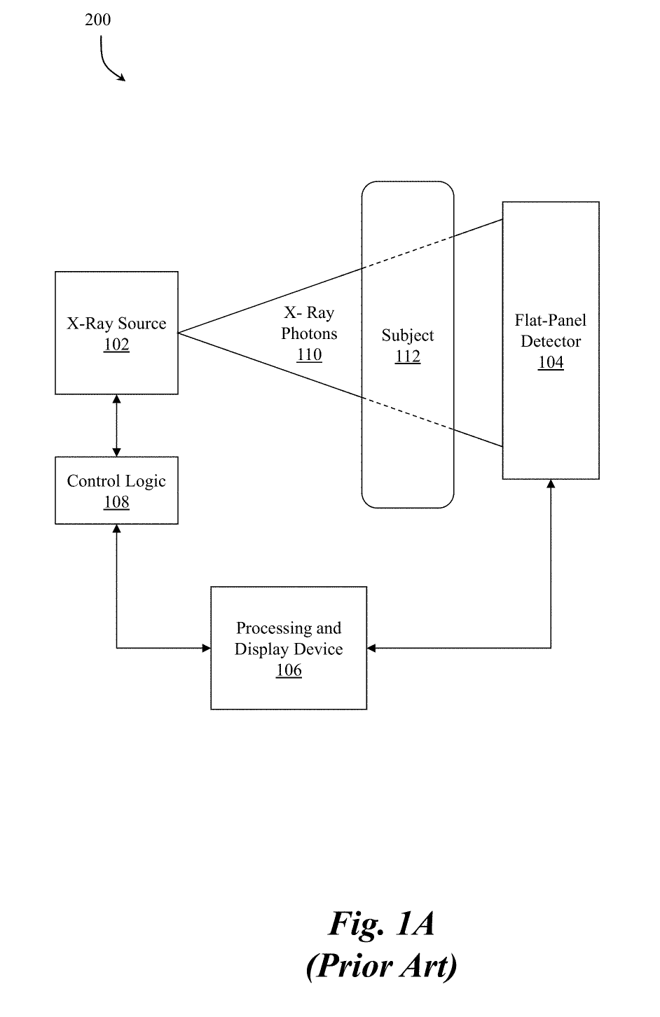

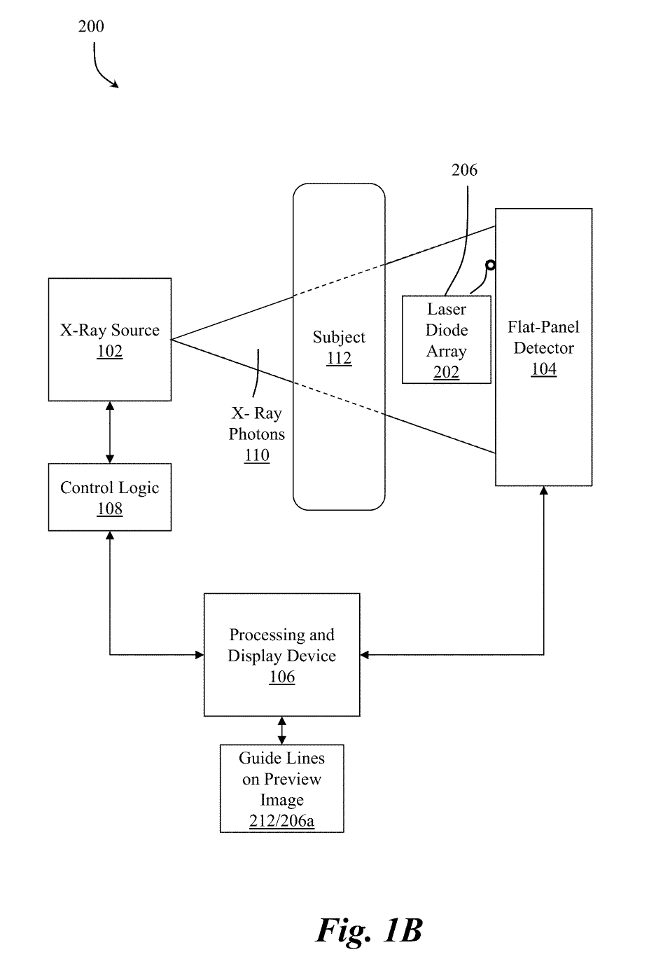

FIGS. 1B through 7, wherein like parts are designated by like reference numerals throughout, illustrate an example embodiment or embodiments of improved operation for a laser guidance system for use in conjunction during a fluoroscopic procedure, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, in a manner still in keeping with the spirit and scope of the present invention.

Traditionally, fluoroscopic imaging procedures can be implemented utilizing any combination of imaging systems (e.g., C-arm, Bi-plane fluoroscopic imager, etc.). FIG. 1A depicts an example implementation of an example imaging system 100 that can be utilized during a fluoroscopic procedure. In particular, FIG. 1A depicts the traditional x-ray photon detection systems 100 implemented for a single plane x-ray application. The x-ray photon detection system 100, as depicted in FIG. 1A, includes an x-ray source device 102, fluoroscopic imager or x-ray photon or radiation detector 104, a processing and display device 106, and a control logic device 108. The x-ray source 102 is a device configured to produce x-ray photons 110 for projection through a subject 112 (e.g., a patient). The x-ray photons 110 can be generated by the x-ray source 102 utilizing any combination of systems and methods known in the art.

The radiation detector 104 is configured to electrically transform the x-ray photons 110 into detectable signals. In particular, in accordance with an example embodiment, the radiation detector 104 is a flat panel detector, which is a thin film transistor (TFT) panel with a scintillation material layer configured to receive energy from visible photons to charge capacitors of pixel cells within the TFT panel. The charges for each of the pixel cells are readout as a voltage data value to the processing and display device 106. The control logic 108 is configured to receive input from the processing and display device 106 and transmit signals to control the x-ray source 102. In particular, the control logic 108 provides signals for operating the x-ray source 102 and when to produce x-ray photons 110. As would be appreciated by one skilled in the art, each of the components within the system 100 can include a combination of devices known in the art configured to perform the imaging tasks discussed herein. For example, an image intensifier is an alternative imaging device that can be utilized in place of the radiation detector 104 system discussed herein without departing from the scope of the present invention.

FIG. 1B depicts an exemplary laser guidance system 200 in accordance with the present invention. In accordance with an example embodiment of the present invention, the laser guidance system 200 includes an x-ray source device 102, an radiation detector 104, a processing and display device 106, and a control logic device 108, as discussed in FIG. 1A. Additionally, the laser guidance system 200 includes one or more rotary laser-diode arrays 202 installed around the fluoroscopic imager (e.g., the x-ray source 102, the radiation detector 104, such as an image intensifier of flat-panel detector). The one or more rotary laser-diode arrays 202 are configured to produce one or more guiding laser beams 206 to be directed onto the subject 112 without exposing the subject 112 to radiation. As would be appreciated by one skilled in the art, any type of fluoroscopic imaging system can be modified to include the one or more rotary laser-diode array 202 without departing from the scope of the present invention. For example, an image intensifier can be similarly mounted and operate in a similar manner as the laser guidance system 200 discussed with respect to FIG. 1B.

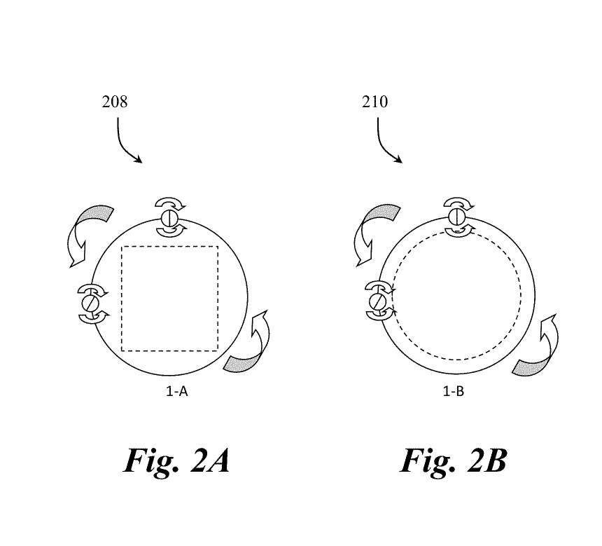

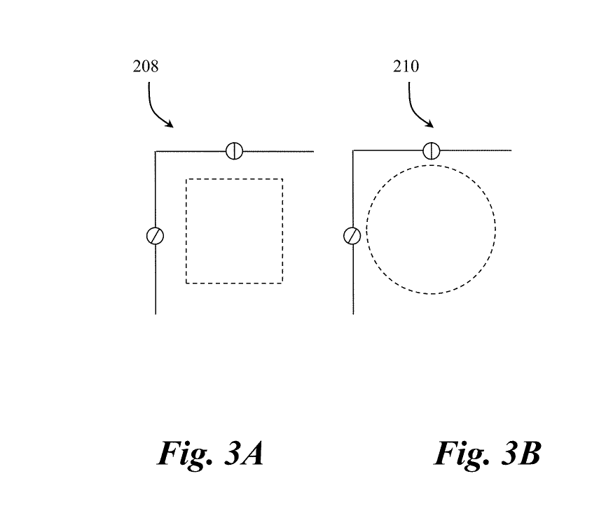

In accordance with an example embodiment of the present invention, each of the laser-diode arrays 202 can be mechanically positioned with an angular coverage (as depicted in FIG. 2A) or a linear translation (as depicted in FIG. 2B). FIG. 2A depicts a laser guidance system 200 with rotary laser diodes 208 and their planetary locus around a fluoroscopic imager with a radiation detector 104 (e.g., a flat panel detector). FIG. 2B depicts a laser guidance system 200 with rotary laser diodes 210 and their planetary locus around a fluoroscopic imager with an image intensifier. As would be appreciated by one skilled in the art, laser diodes can be motorized and rotated aground the x-ray detection device and all laser diodes can be self-rotated around their axis. FIGS. 3A and 3B depict exemplary implementations of the laser guidance system 200 with the laser-diodes 208 and 210 respectively and their respective line locus in two directions in the respective fluoroscopic image for the radiation detector 104 (e.g., FIG. 3A for a flat panel detector and FIG. 3B for an image intensifier). Additionally, the laser diodes can be motorized along rigid tracks, which are mechanically mounted to have a 180 degree coverage of the radiation detector.

During operation, each of the diodes in the laser-diode arrays 202 is configured to emit a fan guide laser beam that appears as a guiding laser beam 206 on the body of the subject 112 as emitted from the laser-diode array 20. In accordance with an example embodiment of the present invention, the laser-diode arrays 202 can be independently and programmatically rotated mechanically, electronically, and/or optically. In particular, the diodes in the laser-diode arrays 202 are positioned and rotated to create the guiding laser beam 206 that corresponds to a plan line planned on a fluoroscopic preview image device (e.g., the processing and display device 106). Each of laser-diodes itself can be programmatically rotated such that beam direction can be properly aligned according to the user specification (e.g., on the preview image 212). For example, surgeons prescribe plan lines on x-ray images and according these line direction and x-ray imaging geometry parameters, the target position and orientation of laser diodes can be determined, aligned and/or rotated.

In accordance with an example embodiment of the present invention, the processing and display device 106 is further configured to provide surgeons with a planning tool to create one or more plan lines 206a on a preview image 212 for the generation of the placement of the guiding laser beam 206 on a subject 112. In particular, surgeons can create the one or more plan lines 206a on a fluoroscopic preview image 212 through a software interface provided by the processing and display device 106. For example, the surgeons can draw on the preview image 212 using a computer mouse, a touch screen input, drawing tablet, or other input device known in the art. The fluoroscopic preview image 212 is based on an image created by any radiation detector fluoroscopic imager, such as an image intensifier or a flat panel detector (as depicted in FIG. 1A). The processing and display device 106 provides a graphical user interface in which the image provided by the radiation detector 104 (e.g., flat panel detector) is provided to the user (e.g., a surgeon) as a preview image 212 for modification. The modification includes the user creating one or more plan lines 206a over the preview image 212 for desired plan lines 206a to be generated by the laser diodes during a procedure (e.g., pre-planning). As would be appreciated by one skilled in the art, the one or more plan lines 206a on the preview image 212 can be planned with any position and line direction that the user desires. The desired plan lines 206a created on the preview image 212 are utilized by the processing and display device 106 to determine the positioning and rotation of the diodes within the laser-diode arrays 202 for display of the guiding laser beams 206 on the subject 112 during the actual procedure. In particular, the one or more plan lines 206a provide the input information, along with x-ray imaging geometry, to compute the positions and orientations of the laser diodes in the laser-diode arrays 202. In accordance with an example embodiment of the present invention, the plan lines 206a are prescribed on preview images through a graphical user interface (GUI) using pointer device or touch screen interaction, this geometrical input data is used to compute the laser diodes within the laser-diode arrays 202 position and orientation.

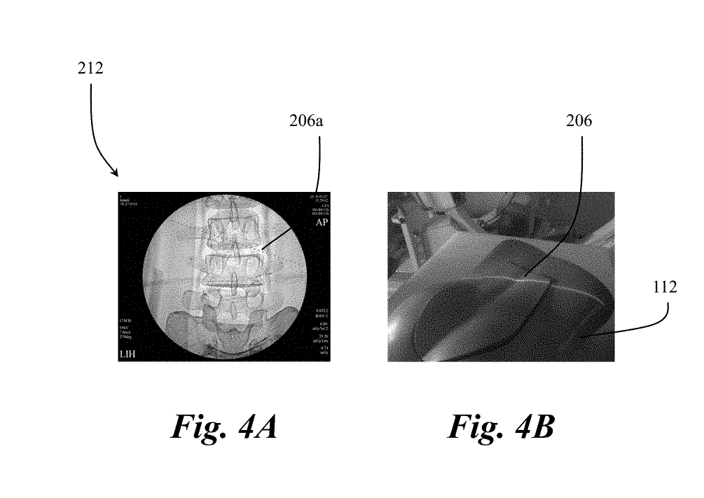

FIG. 4A depicts an example preview image 212 and one or more plan lines 206a viewed on the processing and display device 106. In operation, a user draws desired plan lines 206a on the preview image 212 within a graphical user interface provide by the processing and display device 106. Thereafter, the processing and display device 106 determines a positioning of the diodes of the laser-diode arrays 202 required for directing the guiding laser beams 206 onto the subject 112 at the appropriate positions matching the one or more plan lines 206a. The positioning for the guiding laser beams 206 is provided to the laser-diode arrays 202 such that the diodes can be properly positioned and rotated to provide the corresponding guiding laser beams 206 to be directed onto the subject 112 in the same locations corresponding to the one or more plan lines 206a creates on the preview image 212, as depicted in FIG. 4B. In other words, the one or more plan lines 206a in the software overlay onto the fluoroscopic image are in the same location and positioning as the guiding laser beams 206 produced by the laser-diode arrays 202 as both relate to the subject 112. Thereafter, the surgeon can rely on the guiding laser beams 206 produced by the guiding laser beams 206 during the intra-operative surgery without exposing the subject 112 to additional radiation beyond that which was experienced during the brief snapshot of the fluoroscopic image.

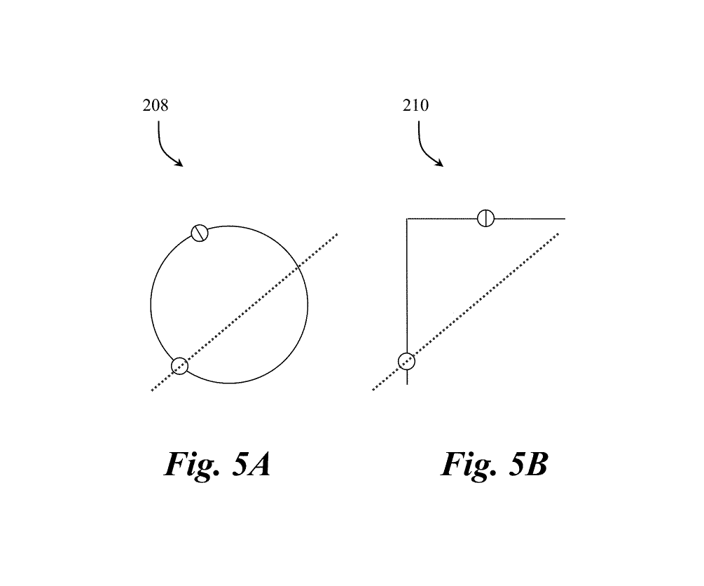

FIGS. 5A and 5B show the one or more laser diodes 208, 210 positioned and aligned according to plan lines 206a created on the fluoroscopic preview image 212. In particular, FIG. 5A depicts the laser diode 208 moved with angular coverage and FIG. 5B depicts the laser diode 210 moved with linear translation with respect to the preview image 212. The laser diodes 210 are positioned and directed onto the subject 112 according to prescribed plan lines 206a on the preview images.

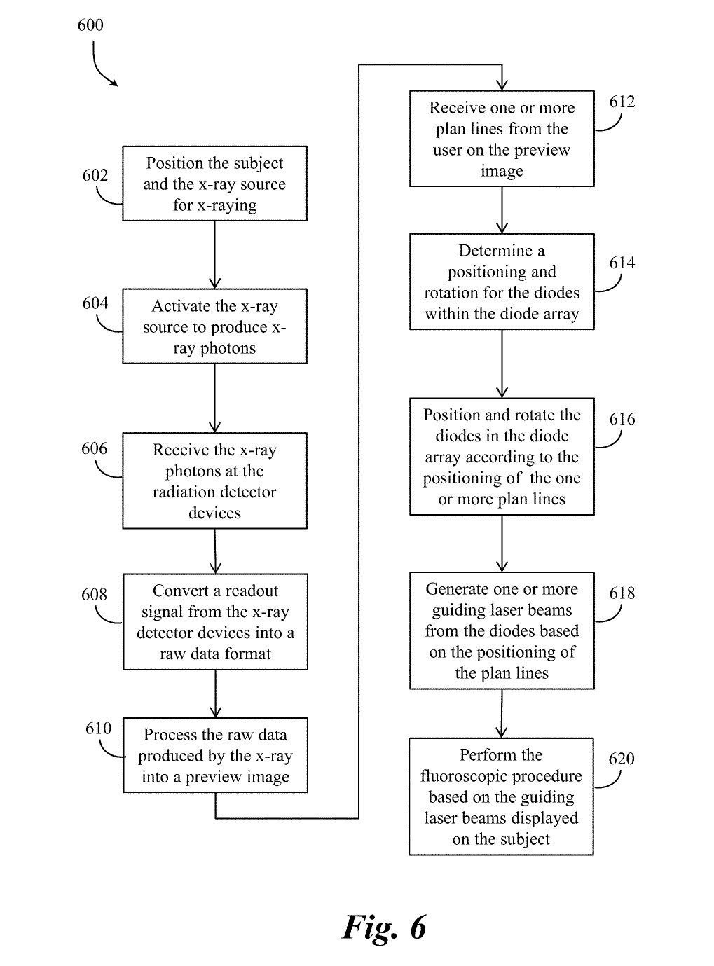

FIG. 6 depicts an example process 600 of operation for the laser guidance system 200 depicted in FIG. 1B and discussed with respect to FIGS. 2A-5B. In particular, FIG. 6 depicts an example implementation of the process 600 to pre-plan the one or more plan lines 206a on a fluoroscopic preview image 212 to be directed as a guiding laser beams 206 (as produced by the laser diode array 202) onto a subject 112. Although, with respect to FIG. 6, the process 600 is discussed with respect to the operation for the laser guidance system 200 depicted in FIG. 1B including a radiation detector 104 system, as would be appreciated by one skilled in the art, the process 600 could be implemented utilizing any other combination of imaging methods and systems (e.g., such as an image intensifier).

At step 602 the subject 112 and the x-ray sources 102 are positioned for performing an x-ray. In particular, the subject 112 and the x-ray sources 102 are positioned in an arrangement to capture the desired area of the subject 112 and the angles of that area desired to be visualized via the x-ray.

At step 604 the x-ray source 102 is activated. In particular, the x-ray source 102 is activated in response to receiving a user instruction input into the processing and display device 106 and transmitted to the x-ray source 102 via the control logic 108. Upon activation, the x-ray source 102 generates x-ray photons 110 in a direction of the subject 112. At step 606 the x-ray photons 110 are absorbed as charges at the radiation detector 104 to be electrically converted for display by the processing and display device 106.

At step 608 the plurality of pixel cells of the radiation detector 104 read the charges stored in the capacitors and provide a readout signal to the processing and display device 106. Thereafter, the processing and display device 106 receives the readout and converts the readout signal from the radiation detector device 104 (e.g., flat-panel detector, x-ray receptor devices, etc.) into a raw data format. In particular, the processing and display device 106 receives the readout signal and converts the signal into a format for display utilizing any system or method known in the art. During this period of time the x-ray can be implemented to have either a continuous or pulsed exposure.

At step 610 the system 100 processes the raw data produced by the x-ray and transforms the raw data into a preview image 212 and displayed on a display device 106 (e.g., a monitor) for interpretation and pre-planning by a user. The display being an x-ray image of the subject 112. At step 612 the processing and display device 106 receives one or more plan lines 206a from a user as an overlay on the displayed preview image 212.

At step 614 the processing and display device 106 determines a positioning and rotation for one more diodes within the laser diode array 202 need to direct guiding laser beams 206 at the same location on the subject 112 in the real world as the one or more plan lines 206a created on the preview image 212. At step 616 processing and display device 106 transmits a signal to the laser diode array 202 (via the control logic 108) position and rotate the diodes in the laser diode array 202 according to determined positioning and rotation based on one or more plan lines 206a.

At step 618 the laser diode array 202 directs one or more guiding laser beams 206 from the diodes in the direction of the subject 112. The one or more guiding laser beams 206 create visible light lines on a surface of the subject 112. Additionally, the guiding laser beams 206 correspond to the location and angle of the one or more plan lines 206a created by the user on the preview image 212 as they related to the locations on the subject 112. At step 620 the user (e.g., a surgeon) can being performing a fluoroscopic procedure based on the guiding laser beams 206 displayed on the subject 112. As a result of the process 600, the laser guidance system 200 provides improved surgical precision while significantly reducing dependence on intra-operative fluoroscopy (e.g., reducing exposure to radiation).

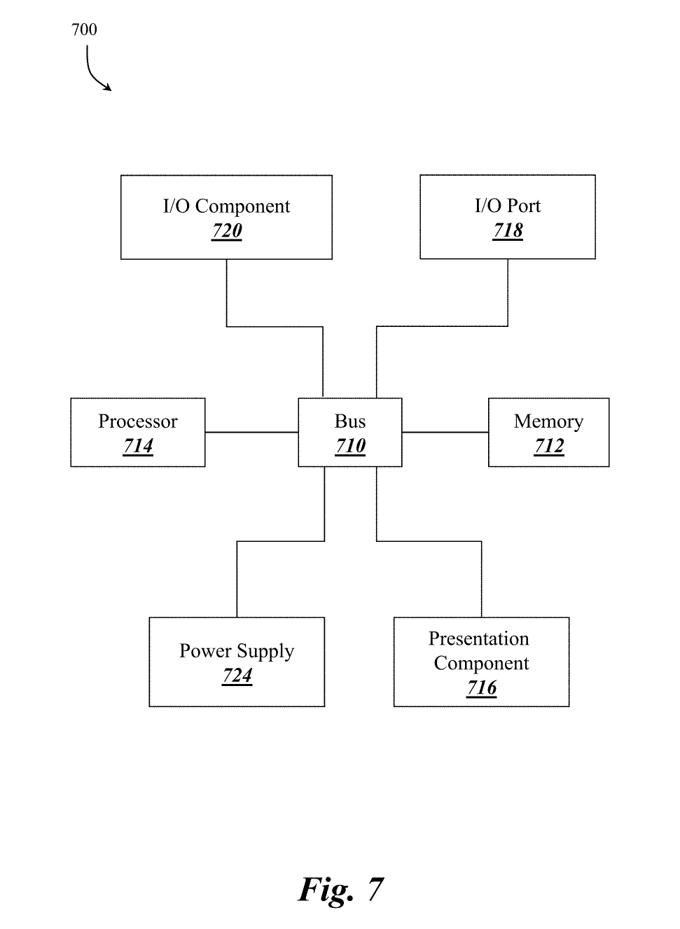

Any suitable computing device can be used to implement the computing devices 106, 108 and methods/functionality described herein and be converted to a specific system for performing the operations and features described herein through modification of hardware, software, and firmware, in a manner significantly more than mere execution of software on a generic computing device, as would be appreciated by those of skill in the art. One illustrative example of such a computing device 700 is depicted in FIG. 7. The computing device 700 is merely an illustrative example of a suitable computing environment and in no way limits the scope of the present invention. A “computing device,” as represented by FIG. 7, can include a “workstation,” a “server,” a “laptop,” a “desktop,” a “hand-held device,” a “mobile device,” a “tablet computer,” or other computing devices, as would be understood by those of skill in the art. Given that the computing device 700 is depicted for illustrative purposes, embodiments of the present invention may utilize any number of computing devices 700 in any number of different ways to implement a single embodiment of the present invention. Accordingly, embodiments of the present invention are not limited to a single computing device 700, as would be appreciated by one with skill in the art, nor are they limited to a single type of implementation or configuration of the example computing device 700.

The computing device 700 can include a bus 710 that can be coupled to one or more of the following illustrative components, directly or indirectly: a memory 712, one or more processors 714, one or more presentation components 716, input/output ports 718, input/output components 720, and a power supply 724. One of skill in the art will appreciate that the bus 710 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, FIG. 7 is merely illustrative of an exemplary computing device that can be used to implement one or more embodiments of the present invention, and in no way limits the invention.

The computing device 700 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 700.

The memory 712 can include computer-storage media in the form of volatile and/or nonvolatile memory. The memory 712 may be removable, non-removable, or any combination thereof. Exemplary hardware devices are devices such as hard drives, solid-state memory, optical-disc drives, and the like. The computing device 700 can include one or more processors that read data from components such as the memory 712, the various I/O components 716, etc. Presentation component(s) 716 present data indications to a user or other device. Exemplary presentation components include a display device, speaker, printing component, vibrating component, etc.

The I/O ports 718 can enable the computing device 700 to be logically coupled to other devices, such as I/O components 720. Some of the I/O components 720 can be built into the computing device 700. Examples of such I/O components 720 include a microphone, joystick, recording device, game pad, satellite dish, scanner, printer, wireless device, networking device, and the like.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may existing in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A medical procedure guidance system, comprising:
a fluoroscopic imaging device comprising a radiation source directed at a radiation detector and configured and arranged to receive a subject patient therebetween;
a laser diode array;
a processing and display device in communication with the fluoroscopic imaging device and the laser diode array;
wherein the fluoroscopic imaging device obtains a fluoroscopic image of the subject patient and communicates the fluoroscopic image to the processing and display device as raw image data;
wherein the processing and display device receives the raw image data and displays a preview image on a display with at least one plan line generated by the processing and display device overlaid onto the preview image; and
wherein the processing and display device instructs the laser diode array to project one or more guiding laser beams onto the subject patient at locations corresponding to the at least one plan line overlaid onto the preview image.

2. The system of claim 1, wherein the radiation detector comprises a thin film transistor (TFT) flat-panel detector with a scintillation material layer.

3. The system of claim 2, wherein the TFT is configured to receive energy from visible photons that charge capacitors of pixel cells within the TFT panel and charges from each of the pixel cells are readout as a voltage data value to the processing and display device.

4. The system of claim 1, wherein the radiation detector comprises an image intensifier configured to readout a voltage data value to the processing and display device.

5. The system of claim 1, wherein the laser diode array comprises a plurality of diodes, each of the plurality of diodes independently positioned and rotated according to a user specification.

6. The system of claim 1, wherein laser-diodes of the laser diode array are mechanically positioned with an angular coverage or linear translation around the fluoroscopic imaging device according to the at least one plan line to direct the laser diode array to project guiding laser beams onto the subject patient.

7. The system of claim 1, wherein the at least one plan line is generated with any position and line direction through the preview image.

8. The system of claim 7, wherein the at least one plan line provides input information and imaging geometry to determine positions and orientations of laser diodes within the laser diode array.

9. A method for utilization of a medical procedure guidance system, the method comprising:
activating a fluoroscopic imaging device comprising a radiation source directed at a radiation detector and configured and arranged to receive a subject patient therebetween;
obtaining, by the fluoroscopic imaging device, a fluoroscopic image of the subject patient
communicating the fluoroscopic image to a processing and display device as raw image data
transforming the raw image data, by the processing and display device, into a preview image of the subject patient;
displaying the preview image on a display with at least one plan line, generated by the processing and display device, overlaid onto the preview image;
instructing, by the processing and display device, a laser diode array to project one or more guiding laser beams onto the subject patient at locations corresponding to the at least one plan line overlaid onto the preview image.

10. The method of claim 9, wherein the at least one plan line is generated in response to receiving user input to create the at least one plan line at a particular orientation and location on the preview image of the subject patient.

11. The method of claim 9, further comprising determining a positioning and rotation of one or more diodes within the laser diode array to generate the one or more guiding laser beams.

12. The method of claim 9, further comprising performing a fluoroscopic procedure relying on the one or more guiding laser beams.
